(19) United States
(12) Patent Application Publication
(10) Pub. No.: US 2006/0178555 A1
(43) Pub. Date: Aug. 10, 2006

(54) Medical antireflux device based on magnets action

(76) Inventor: Mauro Bortolotti, Bologna (IT)

Correspondence Address: BROWDY AND NEIMARK, P.L.L.C.;624 NINTH STREET, NW, SUITE 300, WASHINGTON, DC, 20001-5303, US

(21) Appl. No.: 11/282,744

(22) Filed: Nov. 21, 2005

(30) Foreign Application Priority Data
Feb. 10, 2005 (IT) RE2005A000009

(51) Int. Cl. A61F 2/00; A61F 13/00

(52) U.S. Cl. 600/37

(57) ABSTRACT

An antireflux device is adapted to be surgically applied to the esophagus of a person. The device includes a pair of magnets symmetrically positioned with the opposite polarities of the magnets under the diaphragm of the person so as to compress the lower portion of the esophagus. This closes the lumen to prevent reflux of gastric material into the esophagus. The magnets may be covered with silicon or another biologically inert material.

### FIELD OF THE INVENTION

The field of application of this invention is medical and, precisely, it concerns the prevention of gastroesophageal reflux. The reflux is due to the inability of the lower esophageal sphincter to close the communication between the stomach and the esophagus, in order to prevent the passage in the esophageal lumen of the gastric content, which may cause more or less severe damages to the esophageal mucosa. The aim of the present invention is to prevent the reflux of gastric content into the esophagus.

### PRIOR ART

The problem of preventing the gastroesophageal reflux closing the communication between stomach and esophagus with simple, efficient and devoid of unwanted effects mechanical methods is still unresolved. The most popular procedure, that is the “fundoplication” following Nissen, the Angelchik prosthesis, and the various endoscopic techniques, such as STRETTA, ENDOCINCH, PLICATOR and GATEKEEPER, are insufficient or expose the patient to unwanted side effects and/or complications more or less severe.

### OBJECT AND SUMMARY OF THE INVENTION

The present invention consists of a device represented by two magnets made of plastoferrite or other magnetic material coated with silicon or other biologically inert material, which compress the lower portion of the esophagus, closing the lumen without compromising the blood supply. In a first embodiment magnets can be positioned outside the esophageal wall or in a second embodiment inside the wall, into the thickness of the submucosal layer. In any case the magnets of each device must have such a size and magnetic force that the reciprocal attraction, when they are positioned in the esophagus, generate a pressure of about 10 mmHg inside the esophageal lumen. “In vitro” experiments demonstrated that the force of closure of magnets can be overcome, with consequent opening of the esophageal lumen and passage of gastric content into the esophagus, only by a pressure in gastric cavity more than 10 mmHg. This force of closure, on one hand, is sufficient to prevent the reflux of material from the stomach into the esophagus, and, on the other hand, it allows the passage of the alimentary bolus from the esophagus to the stomach.

Further features and advantages of the present invention will become apparent to those of skill in the art in view of the detailed description of preferred embodiments which follows, when considered together with the attached drawings and claims.

### BRIEF DESCRIPTION OF THE DRAWINGS

FIG. 1 shows a schematic section following a vertical frontal plane of the esophageal zone corresponding to the diaphragm of a person of the first embodiment according to the invention;

FIG. 2 shows a section along section line 2-2 of FIG. 1 through magnets positioned to abut the esophageal wall;

FIG. 3 shows a schematic section following a vertical frontal plane of the second embodiment according to the invention;

FIG. 4 shows a section along section line 4-4 of FIG. 3 that includes the magnets positioned in the esophageal wall;

FIG. 5 shows a section of a tube, technically called “endoscopic overtube” with a bigger lumen through which the endoscope passes to control the right position of the magnets and a smaller lumen through which the magnets are pushed in the esophageal wall, in the second embodiment.

FIG. 6 shows equipment in which in vitro experiments were conducted on the device according to the invention;

FIG. 7 shows intraluminal pressure variations in the segment of the equipment of FIG. 6 connected to the pump and concomitant pressure variations in the segment separated by the magnetic device.

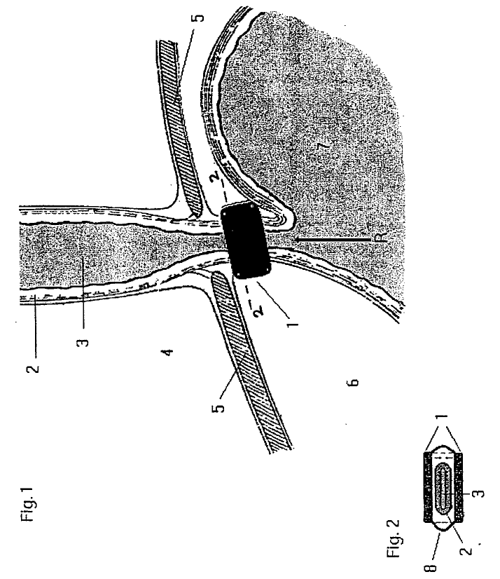

FIGS. 1 and 2 depict the first embodiment of the invention wherein FIG. 1 shows the schematic section following a vertical frontal plane of the esophageal zone in correspondence with the “diaphragm”; whereas the FIG. 2 shows a section following a horizontal plane through the magnets positioned to abut the esophageal wall.

In these figures, the number 1 represents the pair of magnets, 2 the esophageal wall, 3 the esophageal lumen, 4 the thoracic cavity, 5 the diaphragm, 6 the abdominal cavity, and 7 the gastric cavity. The arrow R shows the direction of the reflux, which is to be prevented. 8 represents a couple of loose and adjustable laces, which bind the pair of magnets together, better described below.

In this embodiment, magnets are about 5×20×40 mm in size and are positioned horizontally by means of a laparoscopic or laparotomic procedure, one before, and the other behind the lower portion of the esophagus (cardial zone) with their opposite polarities face to face, so that they attract each other and compress between them the esophagus as indicated in FIG. 1. One of the magnets is fixed behind the esophagus on the posterior wall of the diaphragmatic hiatus by means of four suture points passing through the holes at the four corners of the magnet. The other magnet is positioned in front of the esophagus and fixed to the posterior magnet by four loose and adjustable laces (8) of about 25 mm in length inserted in the holes at the corners of the magnet (FIG. 2).

In this manner, the magnets (1) continuously compress the lower portion of the esophagus closing the lumen and preventing the gastroesophageal reflux. When the patient swallows a bolus of food, the magnets are detached by the pressure generated inside the esophageal lumen by the peristaltic contraction and by the hydrostatic pressure due to the ingested material coming down along the esophagus, with consequent opening of the esophago-gastric passage. After the esophageal content is passed in the stomach, the magnets close again in virtue of their attraction force and intra-abdominal pressure, closing again the communication between the stomach and esophagus.

This embodiment of the device is indicated for patients in whom there are at least 2 cm of the lower esophagus in the abdominal cavity.

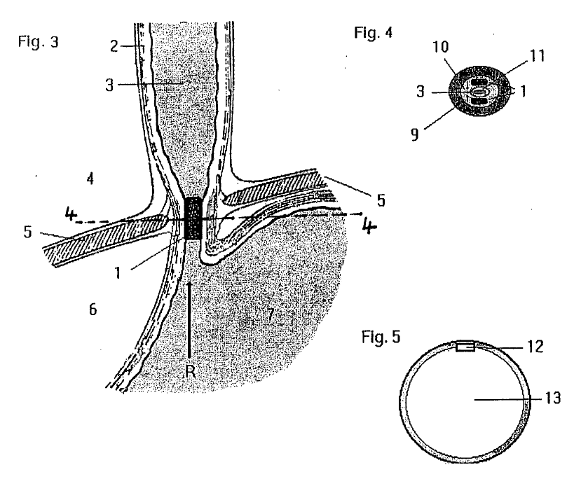

The second embodiment is represented in FIG. 3 as a schematic section following a vertical frontal plane. FIG. 4 shows a detail of the section following a horizontal plane through the magnets (1) positioned in the esophageal wall. FIG. 5 shows a section following a transverse plane through the endoscopic device used to insert the magnets. In FIG. 4, the numbering 1-7 are the same as the other figures, whereas the 9 indicates the esophageal muscular layer, 10 the submucosal layer and 11 the mucosal layer.

In FIG. 5, 12 indicates the opening of the endoscope through which the magnets are pushed to reach their site in the submucosa and 13 indicates the lumen through which the endoscope passes.

In this second embodiment the magnets are about 1.5×5×20 mm in size and are inserted vertically with an endoscopic technique into the submucosal layer of the lower portion of the esophagus. The positioning of the magnets, face-to-face in the esophageal wall, may be carried out by means of the endoscopic overtube commercially available, which is used to position in the esophagus an antireflux prosthesis of expansible material with a similar size. The overtube, however, should be modified by widening to about 2×6 mm the canal (12 of FIG. 5) into which the magnets are pushed to be inserted into the esophageal submucosa previously prepared, the first on one side of the esophageal wall and the second, after a rotation of 180° of the overtube, in the opposite side of the esophageal wall.

The two submucosal magnets work as those of the first embodiment. Once positioned in the esophageal wall, they attract each other closing the passage between esophagus and stomach.

This second embodiment is indicated in all patients with gastroesophageal reflux and may be particularly useful in those in whom 2 cm of the lower portion of the esophagus are not available in the abdominal cavity, as happens when the esophago-gastric junction (cardial) is slipped up in the thoracic cavity and is not possible to replace it in the normal site (slipping hiatal hernia with short esophagus).

The dimension, shape and magnetic force of the magnets in the two embodiments are modifiable in order to be adapted to patients of different build and with different reflux severity.

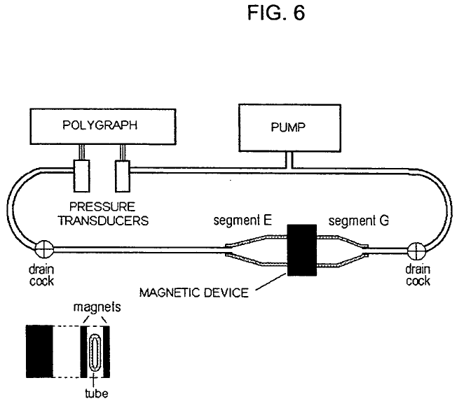

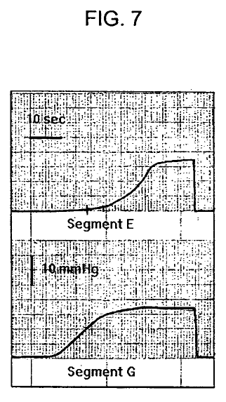

In FIG. 6, showing the apparatus in which the in vitro experiments were conducted, two plastoferrite magnets of 2×4×0.5 cm with a force of 0.16 N/cm² at 7 mm of distance were applied to the opposite sides of a flaccid polyethylene tube mimicking the physical characteristics of the terminal esophagus, as the external diameter was 2.8 cm and the wall was thickened to 3.5 mm by means of a soft plastic material. The two magnets attracting themselves compressed the tube, creating an artificial high pressure zone of 2 cm in length that divide the tube in two segments. Both segments of the tube were connected to pressure transducers and a polygraph. One of the segments was further connected by means of a T tube to a hydraulic pump (segment G of FIG. 6 mimicking the gastric cavity). The pressure was progressively increased in this segment up to a value sufficient to detach the magnets with consequent flowing of the water in the other segment of the tube (segment E of FIG. 6 mimicking the esophageal lumen). As shown in FIG. 7, when the pressure in segment G reached the value of about 11.5 mmHg, the pressure of segment E started to increase and progressively reached a value similar to that of segment G.

When the progressive increase of the pressure in segment G reached an average value of 9.75±1.05 mmHg (mean±SD) the magnets detached allowing a free flow through them and the pressure in segment E started to increase (FIG. 7). Once the pump was stopped and the drains cock opened, the intraluminal pressure decreased and the magnets adhered again closing the passage.

From this experiment, it was determined that a pair of magnets clamping a tube with the characteristics of the distal esophagus is able to give rise to a high pressure zone of about 10 mmHg and 2 cm in length that is considered sufficient to prevent GER (Fein et al.) without blocking the circulation in the vascular bed of the esophageal wall (Guyton and Hall). These studies establish a novel promising approach to resolve the problem of GER treatment.

### REFERENCES

Fein M. Ritter M P, DeMeester T R, Peters J H, Hagen J A, et al., Role of the lower esophageal sphincter and hiatal hernia in the pathogenesis of gastroesophageal disease. Journal of Gastrointestinal Surgery, 1999; 4:405-411.
Guyton, A C, Hall J E, 1966, Textbook of medical physiology. W B Saunders Co., Philadelphia.

1. An antireflux device adapted to be surgically applied to the esophagus of a person comprising:
a pair of magnets (1) symmetrically positioned with opposite polarities of the magnets under the diaphragm of the person compressing the lower portion of the esophagus to close the lumen of the esophagus to prevent reflux of gastric material into the esophagus.

2. The device as in claim 1, wherein each of the magnets is covered with silicon or other biologically inert material.

3. The device as in claim 1, wherein a size, shape and magnetic force of the magnets are modifiable to be adapted to persons of a different build and with different reflux severity.

4. The device as in claim 3, wherein each of the magnets has a size of about 5×20×40, for a person of medium build and develop a pressure of about 10 mm inside the esophageal lumina for a reflux of medium severity.

5. The device as in claim 1, wherein each of the pair of magnets has four holes at corners thereof for suturing a first of the pair of magnets to a posterior wall of the diaphragmatic hiatus of the person and for linking the other second of the pair of magnets to the first with laces.

6. The device as in claim 1, wherein the pair of magnets are linked to each other with loose and adjustable laces (8) of about 25 mm in length.

7. Antireflux device to be positioned endoscopically in the esophageal wall of a person comprising a pair of magnets symmetrically and vertically positioned inside the submucosa of the esophageal wall of the person in correspondence to the cardial zone, with the opposite polarities of the magnets face to face, to compress the inner portion of the esophageal wall of the person to close the lumen thereof to prevent reflux of gastric material into the esophagus.

8. The device as in claim 7, wherein the magnets are coated with silicon or other biologically inert material.

9. The device as in claim 7, wherein a dimension, shape and magnetic force of the pair of magnets are modifiable to be adapted to patients of different build and different reflux severity.

10. The device as in claim 9, wherein the magnets are about 1.5×5.20 mm in size for a person of medium build and develop a pressure of closure of about 10 mm for a condition of medium severity of reflux.
